(19) United States
(12) Patent Application Publication
(10) Pub. No.: US 2022/0069323 A1
(43) Pub. Date: Mar. 3, 2022

(54) METHOD AND APPARATUS FOR ASSESSING THE SUITABILITY OF A FUEL FOR USE IN A FUEL CELL

(71) Applicant: NANOSUN IP LIMITED, Lancaster Lancashire (GB)

(72) Inventors: Joseph HOBBS, Lancaster Lancashire (GB); Graham HODGSON, Lancaster Lancashire (GB)

(21) Appl. No.: 17/415,777

(22) PCT Filed: Dec. 19, 2019
(86) PCT No.: PCT/GB2019/053614

§ 371 Date: Jun. 18, 2021

(30) Foreign Application Priority Data
Dec. 20, 2018 (GB) 1820821.5

(51) Int. Cl. H01M 8/04186; H01M 8/04537; H01M 8/0444; H01M 8/0662; H01M 8/04082; H01M 8/04223; H01M 8/04992; G01N 27/27; G01N 27/407; G01N 33/22

(52) U.S. Cl. CPC H01M 8/04194; H01M 8/04582; H01M 8/04447; H01M 8/0662; G01N 33/225; H01M 8/04231; H01M 8/04992; G01N 27/27; G01N 27/407; H01M 8/04201

(57) ABSTRACT

A method and apparatus for comparing fuel sources to assess the suitability of a fuel for use in a fuel cell. The apparatus comprising an electrochemical sensor comprising a fuel flow channel configured to receive a plurality of input fuels at a plurality of locations along the fuel flow channel. The fuel flow channel configured to supply the plurality of input fuels to an anode of the electrochemical sensor and an electrolyte configured to transmit ionised input fuels from the anode to a cathode of the electrochemical sensor. A control system connected to the electrochemical sensor where the anode and/or the cathode is divided into a plurality of segments and the control system is configured to measure the current in each of the plurality of segments and determine the current density of each of the plurality of segments.

This invention relates to a method and apparatus for assessing the suitability of a fuel for use in a fuel cell. In particular, for comparing the quality of hydrogen fuel streams for use in a fuel cell.

### BACKGROUND

Hydrogen fuel can be used in Proton Exchange Membrane (PEM) fuel cells. PEM fuel cells have several advantages over other types of fuel cells, the fuel cells can: operate at low temperatures; produce no emissions other than water; and rapidly produce power on demand. Recent developments in PEM fuel cells have made them more reliable and closer to commercial deployment in a number of different fields. However, the catalysts used in PEM fuel cells can be vulnerable to poisoning from contaminants present in hydrogen fuel supplied to the cells. The poisoning of the catalyst may reduce the efficiency and cause irreparable damage to the fuel cell. Not all contaminates present in a hydrogen fuel poison the catalyst. Contaminates such as nitrogen and water are inert and do not poison the catalyst.

The production methods of hydrogen fuel may create a fuel containing contaminants capable of poisoning the catalyst in a PEM fuel cell. Hydrogen fuel may be produced from steam reforming of methane which is followed by the separation of contaminants from the fuel using pressure-swing absorption. Alternatively, hydrogen fuel may be produced using electrolysis of water which may create pure hydrogen. Hydrogen fuel may also be formed from the by-products of industrial processes such as metallurgical coke production and the electrolysis of salt.

Once hydrogen fuel has been produced, maintaining the quality of the fuel can be challenging. The storage and transportation of hydrogen fuel can introduce contaminants into the fuel. For example, the transport of hydrogen through carbon steel pipelines can result in methane being formed in the fuel.

It is important to ensure that hydrogen delivered to a PEM fuel cell is of a suitable quality at the point of use in order to reduce the risk of damage to the fuel cell. Current techniques for ensuring hydrogen quality centre on analysing the hydrogen composition to very high tolerances and comparing with a specified standard. Such techniques can be expensive, cumbersome, time-consuming and not suitable for the continuous monitoring of a hydrogen fuel supply.

An example of a hydrogen purification method is described in document U.S. Pat. No. 9,169,118 B1. The method separates hydrogen gas from a gas source using a palladium membrane. The method can create a pure hydrogen gas; however, such membranes are costly and have stringent operating requirements.

An example of a method for protecting a fuel cell is given in document JP 2008243430 A. The document teaches that an impurity monitoring sensor may be located on the upstream side of a hydrogen fuel cell. The impurity monitoring sensor is more sensitive to the impurities in the hydrogen fuel than the fuel cell thereby protecting the fuel cell. However, a disadvantage of this approach is that deterioration of the sensor due to impurities in the fuel may not be easily separated from changes in the sensor performance due to environmental effects such as temperature, humidity, atmospheric pressure or air quality.

An example of a hydrogen purity monitor is described in document GB2497787 A for monitoring hydrogen delivered to a primary stack. The monitor comprises a pair of fuel cells to monitor a hydrogen source where one fuel cell provides a reference cell and the other a test cell. However, this approach results in a complex system comprising both a reference cell and a test cell when components of the cells, such as the air supply and cathode, could be common between the cells leading to a reduction in the cost of the system.

It is an object of the present invention to provide an apparatus for assessing the suitability of a fuel source for use in a fuel cell that overcomes at least some of the disadvantages associated with the prior art. The apparatus may compare hydrogen fuel or any other suitable fuel source.

### BRIEF SUMMARY OF THE DISCLOSURE

In accordance with an aspect of the present invention there is provided an apparatus for comparing fuel sources comprising;

an electrochemical sensor comprising a fuel flow channel configured to receive a plurality of input fuels at a plurality of locations on the fuel flow channel;

the fuel flow channel configured to supply the plurality of input fuels to an anode;

an electrolyte configured to transmit ionised input fuels from the anode to a cathode; and

a control system connected to the electrochemical sensor;

wherein the anode and/or the cathode is divided into a plurality of segments and the control system is configured to measure the current and determine the current density of each of the plurality of segments.

The apparatus may comprise a control circuit connected to each of the plurality of segments and configured to maintain the segments at a constant equipotential. The control system may be configured to compare the quality of the input fuels by detecting differences in the current densities of the plurality of segments. The control system may be configured to produce an output dependent on the quality of the fuel.

In certain embodiments, the plurality of input fuels may share a common fuel source.

The apparatus may comprise a first fuel stream configured to deliver a first input fuel of the plurality of input fuels to the fuel flow channel. The apparatus may comprise a second fuel stream configured to deliver a second input fuel of the plurality of input fuels to the fuel flow channel. The second fuel stream may be configured to deliver the second input fuel to one or more fuel cells.

In certain embodiments, the first fuel stream may comprise a first purifier configured to purify the first input fuel. The first input fuel may be a reference fuel.

In certain embodiments, the apparatus may comprise a component configured to change the concentration of impurities in the second input fuel. The component may be a second purifier. The second purifier may comprise a series of purifiers each configured to remove a different contaminant from the second input fuel.

In certain embodiments, the apparatus may comprise a plurality of intermediate fuel streams extending from a plurality of intermediate positions on the second purifier configured to deliver a plurality of partially purified input fuels to the fuel flow channel. Alternatively or additionally, the apparatus may comprise a plurality of intermediate fuel streams extending from between each of the series of purifiers configured to deliver a plurality of partially purified input fuels to the fuel flow channel.

In certain embodiments, the control system may be configured to prevent delivery of the second input fuel to the one or more fuel cells when the second input fuel has a quality below a specified value.

The apparatus may comprise an oxidizer flow channel adjacent to the cathode. The oxidizer flow channel may comprise a continuous path. The apparatus may comprise a vent configured to remove inert contaminants from the electrochemical sensor.

In certain embodiments, fuel flow channel may be divided into a plurality of sections. In such embodiments, each of the plurality of input fuels may be received by a different section of the fuel flow channel.

In accordance with the present invention there is provided a method for comparing fuel sources comprising:

supplying a plurality of input fuels to a plurality of locations on a fuel flow channel of an electrochemical sensor;

measuring the current produced by the plurality of input fuels in a plurality of segments of an anode or a cathode of the electrochemical sensor; and

determining the current density in each of the plurality of segments.

In certain embodiments, the method may comprise detecting differences in the current densities of the plurality of segments to compare the quality of the plurality of input fuels. The method may comprise producing an output dependent on the quality of the input fuels. Detecting differences in the current densities of the plurality of segments to compare the quality of the plurality of input fuels may comprise using rule base programming methods or machine learning techniques.

In accordance with an aspect the present invention there is provided an apparatus for comparing air sources comprising;

an electrochemical sensor comprising a fuel flow channel configured to receive an input fuel;

the fuel flow channel configured to supply the input fuel to an anode;

an electrolyte configured to transmit ionised input fuel from the anode to a cathode;

an oxidizer flow channel configured to supply a plurality of input air streams to the cathode;

the oxidizer flow channel configured to receive the plurality of input air streams at a plurality of locations on the oxidizer flow channel; and

a control system connected to the electrochemical sensor;

wherein the anode and/or the cathode is divided into a plurality of segments and the control system is configured to measure the current and determine the current density of each of the plurality of segments.

The apparatus may comprise a control circuit connected to each of the plurality of segments and configured to maintain the segments at a constant equipotential. The control system may be configured to compare the quality of the air streams by detecting differences in the current densities of the plurality of segments. The control system may be configured to produce an output dependent on the quality of the air streams.

In certain embodiments, fuel flow channel may comprise a continuous path. The oxidizer flow channel may be divided into a plurality of sections. In such embodiments, each of the plurality of input air streams may be received by a different section of the oxidizer flow channel.

### BRIEF DESCRIPTION OF THE DRAWINGS

Embodiments of the invention are further described hereinafter with reference to the accompanying drawings, in which:

FIG. 1 schematically shows a fuel comparison apparatus according to an embodiment of the present invention;

FIG. 2 schematically shows an electrochemical sensor according to an embodiment of the present invention;

FIG. 3 schematically shows an electrochemical sensor according to an alternative embodiment of the present invention;

FIG. 4 schematically shows an electrochemical sensor according to an alternative embodiment of the present invention;

FIG. 5 schematically shows a fuel comparison apparatus according to another embodiment of the present invention;

FIG. 6 schematically shows a fuel comparison apparatus according to another embodiment of the present invention;

FIG. 7 schematically shows a fuel comparison apparatus according to another embodiment of the present invention; and

FIG. 8 schematically shows a fuel comparison apparatus according to another embodiment of the present invention.

### DETAILED DESCRIPTION

Embodiments of the present invention provide a method and apparatus for assessing the suitability of a fuel source for use in a fuel cell. The apparatus may be used to reduce the number of contaminants in fuel. Although the present invention may be used to compare hydrogen fuels, the apparatus may also be used to compare any suitable fuel type.

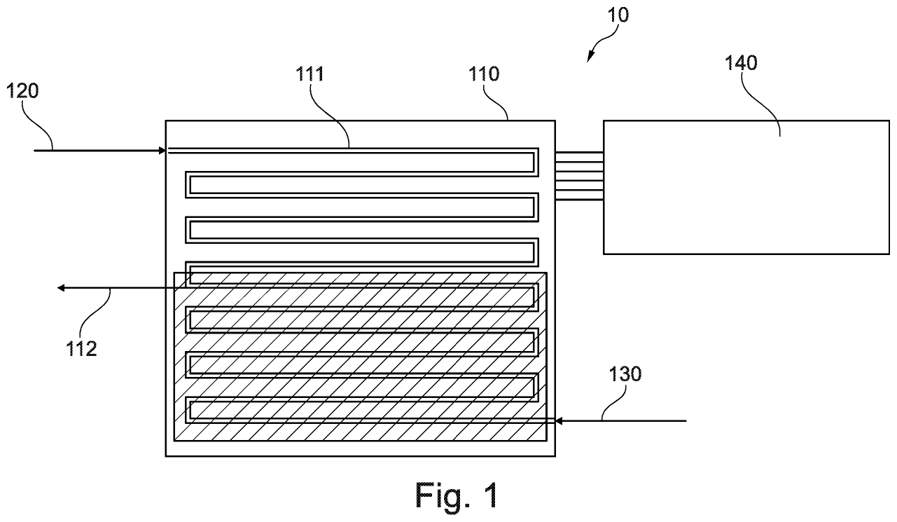

FIG. 1 shows a fuel comparison apparatus 10 according to an embodiment of the invention. The fuel comparison apparatus 10 comprises an electrochemical sensor 110 connected to a control system 140.

In the embodiment shown in FIG. 1, the electrochemical sensor 110 comprises a fuel flow channel 111. The fuel flow channel 111 is configured to receive a first input fuel and a second input fuel at different locations on the fuel flow channel 111. A first fuel stream 120 may be configured to deliver the first input fuel to the fuel flow channel 111. A second fuel stream 130 may be configured to deliver the second input fuel to the fuel flow channel 111. The first and second input fuels may contain different levels of contaminants. For example, the first input fuel may be a reference fuel suitable for use in a fuel cell and the second input fuel may be a fuel of unknown quality. The fuel flow channel 111 may comprise an elongate path. The path may be a serpentine path or any other suitable shape.

The electrochemical sensor 110 comprises an anode (not shown in FIG. 1). The fuel flow 111 channel may be adjacent to the anode. The fuel flow channel 111 is configured to supply the first and second input fuels to the anode. The input fuels are delivered to different locations on the fuel flow channel 111 as shown in FIG. 1. As such, when in use, the first input fuel will make contact with a different part of the anode to the second input fuel.

According to this embodiment, the anode of the electrochemical sensor is divided into a plurality of segments. When in use, each segment of the anode is maintained at a constant equipotential. The fuel comparison apparatus 10 may comprise a series of control circuits, connected to each segment of the anode. The control circuits may be configured to maintain each segment of the anode at the constant equipotential.

When fuel is delivered to the electrochemical sensor 110, each segment of the anode will be supplied with fuel from a different location along the fuel flow channel 111. As such, each segment of the anode may receive the first input fuel, the second input fuel or a combination of the first and the second input fuels. To improve the performance of the fuel comparison apparatus 10, the fuel flow channel 111 may be divided into a plurality of sections. The first and the second input fuels may be delivered to different sections of the fuel flow channel 111. The sections may be separated so that fuel cannot flow from one section of the fuel flow channel 111 to another section. As such, the segments of the anode may receive either the first input fuel or the second input fuel and not a combination of input fuels. Dividing the fuel flow path 111 into sections may improve the ability of the fuel comparison apparatus 10 to discern between the different input fuels.

The electrochemical sensor 110 is configured so that when input fuels are supplied to the segments of the anode, the anode ionizes the fuel, liberating electrons from the input fuel. The electrochemical sensor 110 comprises an electrolyte to transmit the ionized fuel from the anode to a cathode of the electrochemical sensor 110. The electrolyte may be located between the anode and the cathode. The type of electrolyte may vary depending on fuel type supplied to the electrochemical sensor 110. The electrochemical sensor 110 also comprises an external circuit configured to conduct a current, comprising the electrons liberated from the fuel at the anode, from the anode to the cathode. The liberated electrons, ionized fuel and an oxidizer combine at the cathode to form the waste products of the electrochemical sensor 110. The electrochemical sensor 110 may comprise an oxidizer flow channel configured to deliver the oxidizer to the cathode. The oxidizer flow channel may be located adjacent to the cathode. The oxidizer flow channel is configured to receive an oxidizer from a single source. Additionally, the electrochemical sensor may comprise a vent 112 to purge (e.g. periodically) inert contaminates, such as nitrogen and water, from the fuel flow path 111 of the electrochemical sensor 110.

The control system 140 is configured to measure the current produced in each segment of the anode. The control system 140 may be configured to determine the current density in each segment of the anode. The current density in each segment of the anode depends, at least in part, on the quality of the fuel in supplied to the segment. The electrochemical sensor 110 comprises a catalyst to facilitate the ionization of the input fuels at the anode. Contaminates present in the first and second input fuels poison the catalyst at the location where the contaminated fuel makes contact with the catalyst. The poisoning of the catalyst leads to a reduction in the current generated in the poisoned part of the electrochemical sensor 110. For example, if the first input fuel contains no contaminates (i.e. is a reference fuel) and the second fuel input contains contaminates, the first input fuel would produce a higher current density at the anode compared to the second input fuel. As such, the differences in the current densities in the segments of the anode provide an indication of the quality of the fuel supplied to that segment of the anode.

The control system 140 may be configured to detect differences in the current densities (in respect of the segments) and thereby determine differences in the quality of the first and second input fuels. The control system 140 may use rule based programming methods or machine learning to compare the current densities. Alternatively, any other suitable technique may be used. If the first input fuel is a reference fuel and the second input fuel may be a fuel unknown quality, the comparison of the current densities by the control system 140 may indicate whether the second input fuel is suitable for use in a fuel cell.

The control system 140 may be configured to produce an output which may indicate the quality of one or both of the first and second input fuels. The output may comprise a warning indicating poor fuel quality of one of the input streams. The output may comprise a warning indicating that an input fuel is unsuitable for use in a fuel cell. If the control system determines that either input fuel is unsuitable for use in a fuel cell, the control system 140 may be configured to prevent the input fuels from being supplied to a fuel cell.

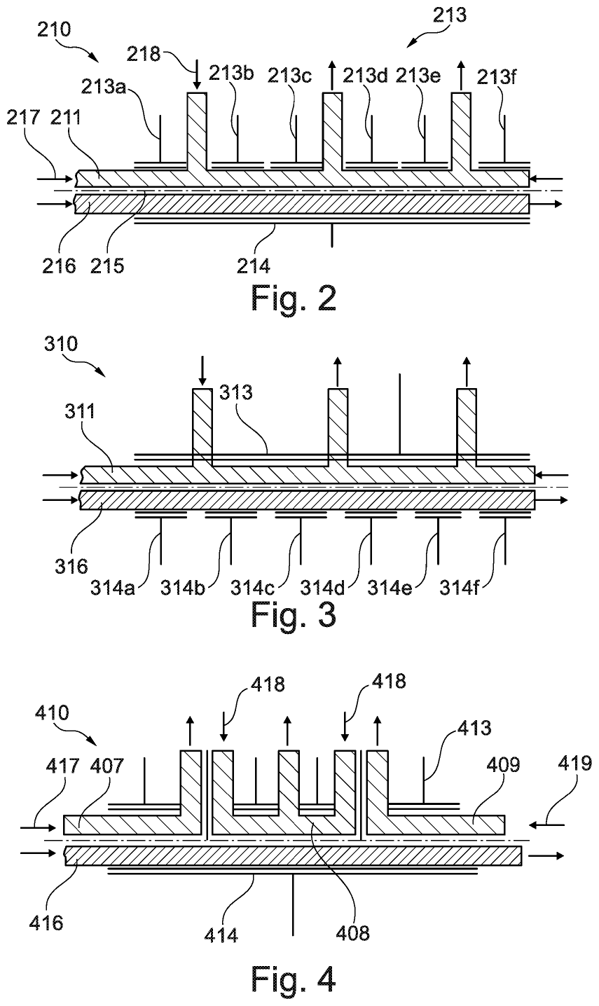

The fuel comparison apparatus 10 may comprise an electrochemical sensor 211 according the embodiment schematically shown in FIG. 2. Reference numerals in FIG. 2 correspond to those used in FIG. 1 with respect to alike component and features, but are transposed by 100. The electrochemical sensor 210 comprises an anode 213 divided into a plurality of segments 213a, 213b, etc. and a single (non-segmented) cathode 214. The fuel flow channel, adjacent to the anode 213, is configured to receive a first input fuel 217 and a second input fuel 218. The oxidizer flow channel 216, adjacent to the cathode 214, is configured to receive an oxidizing gas. The electrolyte 215 transmits ionized fuel from the anode to the cathode.

In an alternative embodiment, the electrochemical sensor 310 schematically shown in FIG. 3 may be used in the fuel comparison apparatus 10. Reference numerals in FIG. 3 correspond to those used in FIG. 2 with respect to alike component and features, but are transposed by 100. In the embodiment shown in FIG. 3, the electrochemical sensor 310 comprises a single (non-segmented) anode 313 and a cathode which is divided into a plurality of segments 314a, 314b, 314c etc. In general, ions produced at the anode travel to the cathode along a trajectory substantially perpendicular to the anode and cathode. Therefore, the electrochemical sensor 310 may be used in a fuel apparatus similar to that described above with reference to FIG. 1. The fuel comparison apparatus 10 would be modified such that the segments of the cathode are maintained at the constant equipotential rather than the anode. Furthermore, the control system 140 would be configured to measure the current produced in each segment 314a, 314b, 314c etc. of the cathode and determine the current density in each segment 314a, 314b, 314c etc. of the cathode.

The comparison of input fuels using the fuel comparison apparatus 10 requires only one of the anode or the cathode to be divided into a plurality of segments. However, to improve the performance of the fuel comparison apparatus 10 additional components of the electrochemical sensor may be divided into segments or sections. Both the cathode and the anode of the electrochemical sensor may be divided into a plurality of segments. The segmentation of the anode and the cathode may be substantially identical to one another. The electrolyte may be divided into a plurality of segments. When only one of the anode or the cathode is divided into a plurality of segments, the segmentation of the electrolyte may be substantially identical to that of the segmented electrode. When both the anode and the cathode are divided into a plurality of segments, the segmentation of the electrolyte, anode and cathode may be substantially identical to one another. As discussed above, the fuel flow channel 111 may also be divided into a plurality of sections. Each input fuel may be delivered to a different section of the fuel flow channel. Although the oxidizer flow channel may be divided into a plurality of sections preferably the oxidizer flow channel comprises a continuous channel to deliver an oxidizer uniformly to the cathode. Additional segmentation of the electrochemical sensor may improve the ability to measure local current densities or to discern between the multiple gas streams. However, additional segmentation of the electrochemical sensor may increase the manufacturing costs of the electrochemical sensor.

In the embodiment described above with reference to FIG. 1, the fuel comparison apparatus 10 is configured to compare two input fuels. However, in alternative embodiments, the electrochemical sensor 110 may be configured to receive more than two input fuels from more than two fuel streams. Each input fuel may contain different levels of contaminants. The fuel comparison apparatus 10 may be configured to receive each input fuel at a different location on the fuel flow channel 111. As such, the input fuels may be supplied to different segments of the anode or cathode. The fuel flow channel 111 may be divided into sections. An example of the division of the fuel flow channel with three input fuels is schematically shown in FIG. 4. Each input fuel 417, 418, 419 may be received by a different section 407, 408, 409 of the fuel flow channel 411. The sections 407, 408, 409 may be separated so that fuel cannot flow from one section of the fuel flow channel 411 to another section. In the same way as described above, the control system 140 may determine differences in the input fuels by comparing the current densities in the segments of the anode or cathode. The control system 140 may produce an output depending on the quality of one or more of the input fuels. Examples of embodiments where two or more input fuels are received by the electrochemical sensor 110 are described below.

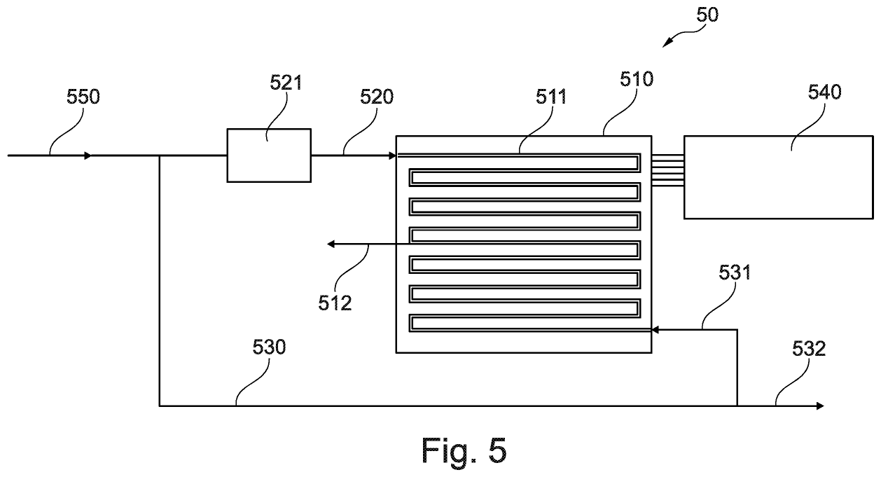

FIG. 5 shows an alternative embodiment of a fuel comparison apparatus 50. The fuel comparison apparatus 50 comprises an electrochemical sensor 510 connected to a control system 540. Reference numerals in FIG. 5 correspond to those used in FIG. 1 with respect to alike component and features, but are transposed by 400.

The fuel comparison apparatus 50 comprises a first fuel stream 520 and a second fuel stream 530 that may share a common fuel source 550. The first fuel stream 520 is configured to deliver the first fuel to the fuel flow channel 511 of the electrochemical sensor 510. The first flow stream 520 may comprise a purifier 521 which modifies fuel from the fuel source 550 to produce the first input fuel. The purifier 521 may be configured to remove contaminants from the first input fuel. The purifier 521 may create a reference fuel stream wherein the fuel contains a negligible number of contaminants that may poison the catalyst in the electrochemical sensor 510. The purifier 521 may leave inert contaminants, not harmful to the catalyst, in the fuel. The purifier 521 may comprise a palladium purifier or any other suitable purifier.

The second fuel stream 530 is configured to deliver the second input fuel to the fuel flow channel 511 of the electrochemical sensor 510. The second flow channel 530 may be configured to split into two streams 531, 532 to deliver the second input fuel to the fuel flow channel 511 and to deliver the second input fuel to one or more fuel cells. The second input fuel may deliver unmodified fuel from the fuel source 550 to the electrochemical sensor 510.

As described above, the control system 540 is configured to compare the current densities created in the segments of the anode by the first and second input fuels. The current densities produced in the electrochemical sensor 511 by the first and second input fuels may differ because the second input fuel may contain contaminates that are removed from the first input fuel by the purifier 521.

The control system 540 may be configured to identify the quality of the second input fuel relative to the first input fuel. If the first input fuel is a reference fuel, the comparison of the first and second input fuels by the control system may indicate the suitability of the second input fuel for use in the one or more fuel cells.

The control system 540 may be configured to produce a warning indicating the quality of the second fuel and whether the second input fuel may damage the one or more fuel cells. Additionally, if the second input fuel may damage the one or more fuel cells, the control system 540 may be configured to automatically shut down the supply of fuel 532 to the one or more fuel cells. This would reduce the chance of significant or irreversible damage being done to the one or more fuel cells.

The use of a single fuel source 550 and the on-location creation of a reference stream is advantageous because it accounts for variations from environmental factors that may alter the current density produced by a fuel in the electrochemical sensor 510. Additionally, the automatic shut down of poor quality fuel to the one or more fuel cells reduces the risk of damage to the cells and therefore reduces operational costs.

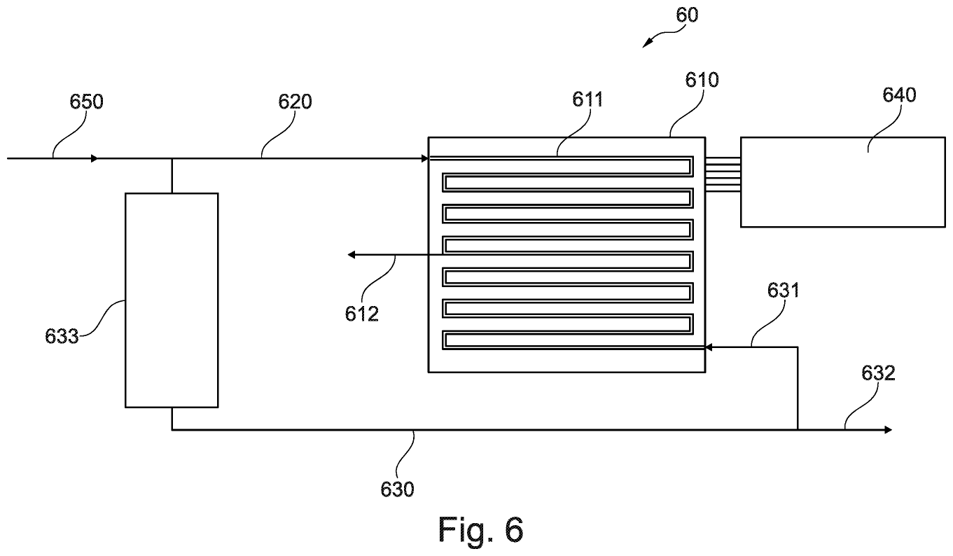

FIG. 6 shows an alternative embodiment of a fuel comparison apparatus 60. The fuel comparison apparatus 60 comprises an electrochemical sensor 610 connected to a control system 640. Reference numerals in FIG. 5 correspond to those used in FIG. 1 with respect to alike component and features, but are transposed by 500.

The fuel comparison apparatus 60 comprises a first fuel stream 620 and a second fuel stream 630 that share a common fuel source 650. The second fuel stream 630 is configured to deliver the second input fuel to the fuel flow channel 611 of the electrochemical sensor 610. The second fuel stream comprises a component 633 configured to modify the number of contaminates in the fuel supplied from the fuel source 650. The modification of fuel from the fuel source 650 by the component 633 produces the second input fuel. The component 633 may be a purifier, compressor, pipeline, storage facility or any suitable component capable of modifying the number of contaminates in the fuel (either intentionally or otherwise). The operation or the failure of the component 633 may change the number of impurities in the fuel. The second fuel stream 630 is configured to provide the second input fuel to the electrochemical sensor 610 and may provide the second input fuel to one or more fuel cells.

The first input fuel stream 620 is configured to deliver fuel from the fuel source 650 to the sensor. The first input fuel may comprise unmodified fuel from the fuel source 650.

As described above, the control system 640 is configured to compare the current densities created in the segments of the anode and/or cathode by the first and second input fuels. The current densities produced in the electrochemical sensor 611 by the first and second input fuels may differ due to the effect of the component 633. As such, the quality of the first and second input fuels may be compared and the performance of the component 633 over time may be monitored.

The control system 640 may be configured to produce a warning indicating the quality of the input fuels. Additionally, if the second input fuel may damage the one or more fuel cells, the control system 640 may be configured to automatically shut down the supply of fuel to the one or more fuel cells. This would reduce the chance of significant or irreversible damage being done to the one or more fuel cells.

In one embodiment the component 633 may be a purifier. The purifier may comprise absorbent and catalytic materials. A purifier may be used in the second fuel stream 630 when the fuel from the fuel source 650 requires upgrading to a suitable quality before delivery to the one or more fuel cells. In use, the control system 640 may be configured to monitor the current densities produced in the electrochemical sensor 610 by the first and second input fuels. Over time the purifier performance may decrease. The decrease in performance may affect the current densities produced in the electrochemical sensor 610 by the first and second input fuels. The control system 640 may produce a warning when the performance of the purifier has reduced. The control system 640 may be configured to automatically shut down the supply of fuel to the one or more fuel cells when the performance of the purifier has reduced.

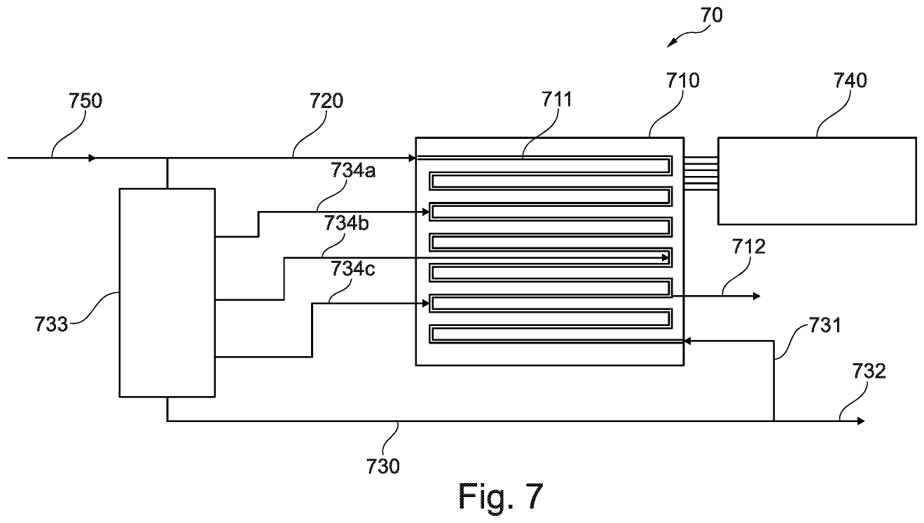

The fuel comparison apparatus 60 may be modified as illustrated in an embodiment shown in FIG. 7. FIG. 7 shows a fuel comparison apparatus 70 in accordance with a further alternative embodiment of the present invention. Reference numerals in FIG. 7 correspond to those used in FIG. 6 with respect to alike component and features, but are transposed by 100.

The fuel comparison apparatus 70 comprises a component 733 in the second fuel stream 730. The component 733 is configured to modify the number of contaminants in the fuel provided by the fuel source 750 to produce the second input fuel. As discussed above in relation to the embodiment shown in FIG. 6, the component 733 may be a purifier, compressor, pipeline, storage facility or any suitable component capable of modifying the number of contaminates in the fuel.

The fuel comparison apparatus 70 comprises a plurality of intermediate fuel streams 734a, 734b, 734c. The intermediate fuel streams 734a, 734b, 734c extend from intermediate points along the component 733. The intermediate fuel streams 734a, 734b, 734c are configured to deliver fuel that has passed through a part of the component 733 to the fuel flow channel 711 of the electrochemical sensor 710. The fuel flow channel 711 is configured to receive the first and second input fuels and the plurality of partially modified fuels at different locations on the fuel flow channel 711. As such, each fuel may be delivered to a different segment of the anode. Alternatively, the ionized fuel from each input fuel may be transmitted through the electrolyte to a different segment of the cathode. The embodiment in FIG. 7 shows three intermediate streams 734a, 734b, 734c but any number of intermediate streams may be used.

As described above, the control system 740 is configured to compare the current densities created in the segments of the anode and/or cathode by the first input fuel, the second input fuel and the intermediate input fuels. The current densities produced in the electrochemical sensor 711 by the input fuels may differ due to the effect of the component 733. The control system 740 may be configured to compare the quality of the first input fuel, the second input fuel and the plurality of partially purified fuels. The control system 740 may produce an output depending on the quality of one or more of the input fuels. Additionally, if the second input fuel may damage the one or more fuel cells, the control system 740 may be configured to automatically shut down the supply of fuel to the one or more fuel cells.

In an embodiment where the component 733 is a purifier, the fuel comparison apparatus 70 enables the loading of the purifier to be determined by analyzing the fuel quality of the intermediate fuels compared to the unpurified fuel (i.e. the first input fuel) and the fully purified fuel (i.e. the second input fuel). This is advantageous because it enables the lifetime of the purifier to be estimated and indicates when the purifier should be replaced thereby reducing the risk of damage to the one or more fuel cells.

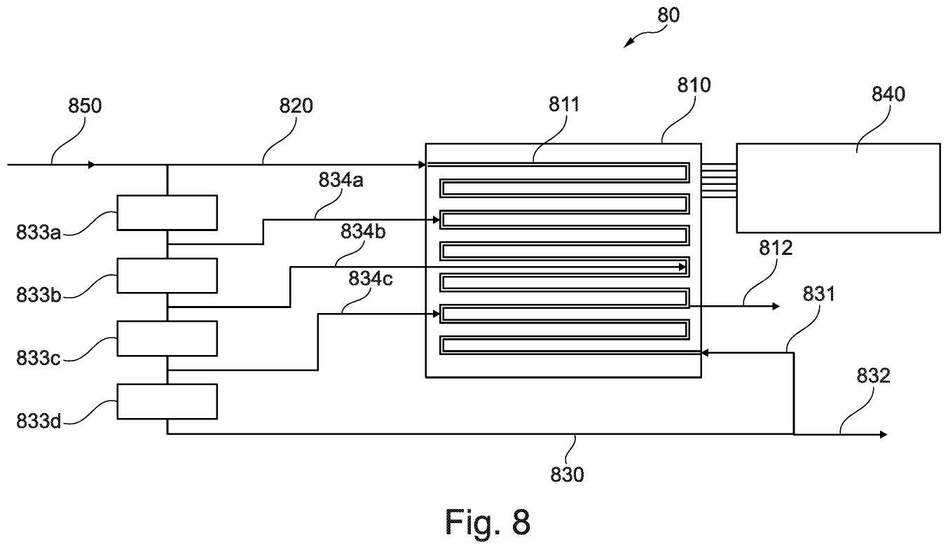

The fuel comparison apparatus 60 shown in FIG. 6 may be modified as illustrated in an embodiment of FIG. 8. FIG. 8 shows a fuel comparison apparatus 80 in accordance with a further alternative embodiment of the present invention. Reference numerals in FIG. 8 correspond to those used in FIG. 6 with respect to alike component and features, but are transposed by 200.

The fuel comparison apparatus 80 comprises a plurality of purifiers 833a, 833b 833c, 833d in the second fuel stream 830. Each purifier may be configured to remove a different contaminate from the fuel provided by the fuel source 850. The purifiers 833a, 833b 833c, 833d may comprise absorbent materials to remove specific impurities. The fuel comparison apparatus 80 comprises a plurality of intermediate fuel streams 834a, 834b, 834c. The intermediate fuel streams 834a, 834b, 834c extend from between each purifier of the plurality of purifiers 833a, 833b 833c, 833d to the electrochemical sensor 810. The intermediate fuel streams 834a, 834b, 834c are configured to deliver partially purified fuel to the fuel flow channel 811 of the electrochemical sensor 810. The fuel flow channel 811 is configured to receive the first and second input fuels and the plurality of partially purified fuels at different locations on the fuel flow channel 811. As such, each fuel is delivered to a different segment of the anode. Alternatively, the ionized fuel from each input fuel may be transmitted through the electrolyte to a different segment of the cathode. The embodiment in FIG. 8 shows four purifiers 833a, 833b 833c, 833d and three intermediate streams 834a, 834b, 834c however, any number of intermediate streams may be used.

As described above, the control system 840 is configured to compare the current densities created in the segments of the anode and/or cathode by the first input fuel, the second input fuel and intermediate input fuels. The current densities produced in the electrochemical sensor 811 by the fuels may differ due to the removal of contaminates by the purifiers 833a, 833b 833c, 833d. The control system 840 may be configured to compare the quality of the first input fuel, the second input fuel and the plurality of partially purified fuels. The control system 840 may produce an output depending on the quality of one or more of the input fuels. Additionally, if the second input fuel may damage the one or more fuel cells, the control system 840 may be configured to automatically shut down the supply of fuel to the one or more fuel cells.

The embodiment illustrated in FIG. 8 enables the control system to determine the differences in the rate of decay of the current density from each of the plurality of purifiers. As such, a semi-quantitative concentration for the contaminates targeted by each of the plurality of purifiers 853a, 853b 853c may be determined. The embodiment illustrated in FIG. 8 provides a cost effective and compact hydrogen analyzer. The analyzer may provide a semi-quantitative measure of the concentration of specific impurities in a hydrogen fuel supply that adversely affects the performance of a fuel cell. Information about what contaminates are present in a fuel source enables specific purifiers to be used for certain fuel sources which may reduce operational costs.

The embodiments described with reference to FIG. 6, 7 or 8 may be modified to encompass the reference fuel stream as described with reference for FIG. 5. As such, the control system may compare any input fuel with a fuel source that contains a negligible number of contaminants which may poison the catalyst in the electrochemical sensor.

In the embodiments described above, the electrochemical sensor may be integrated with one or more additional electrochemical sensors. Different input fuels may be received by different sensors.

The above described fuel comparison apparatus and electrochemical sensor are configured to compare the quality of different input fuels. In an alternative embodiment, the apparatus and electrochemical sensor may be configured to compare the air quality of different air sources. In such an embodiment, the oxidizer flow channel is configured to receive a plurality of input air streams. Each input air stream may be from a different air source. Each air source may contain air comprising a different number of contaminants. The oxidizer flow channel may be divided into a plurality of sections. Each air stream may be received by a different section of the oxidizer channel. The anode, cathode and electrolyte may be divided into segments as described above for the fuel comparison apparatus 10. However, only one of the anode or the cathode are required to be segmented. When in use, each segment of the anode or cathode is maintained at a constant equipotential. The air streams are delivered to different locations on the oxidizer flow channel 111. As such, when in use, the different air streams make contact with different parts of the cathode. A single fuel source is delivered to the fuel flow channel. In certain embodiments, the fuel flow channel comprises a continuous path, not divided into sections, to deliver fuel uniformly to the anode. In the same way as described above, the control system 140 may be configured to measure the current produced in each segment of the anode and/or cathode and determine the current density in each segment of the anode and/or cathode. The control system 140 may be configured to detect differences in the current densities (in respect of the segments) and thereby determine differences in the quality of the input air streams supplied to the electrochemical sensor.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. An apparatus for comparing fuel sources comprising:
an electrochemical sensor comprising a fuel flow channel configured to receive a plurality of input fuels at a plurality of locations on the fuel flow channel;
the fuel flow channel configured to supply the plurality of input fuels to an anode;
an electrolyte configured to transmit ionized input fuels from the anode to a cathode; and
a control system connected to the electrochemical sensor;
wherein the anode or the cathode is divided into a plurality of segments and the control system is configured to measure the current and determine the current density of each of the plurality of segments.

2. The apparatus of claim 1, comprising a control circuit connected to each of the plurality of segments and configured to maintain the segments at a constant equipotential.

3. The apparatus of claim 1, wherein the control system is configured to compare the quality of the input fuels by detecting differences in the current densities of the plurality of segments.

4. The apparatus of claim 3, wherein the control system is configured to produce an output dependent on the quality of the fuel.

5. The apparatus of claim 1, wherein the plurality of input fuels share a common fuel source.

6. The apparatus of claim 1, comprising a first fuel stream configured to deliver a first input fuel of the plurality of input fuels to the fuel flow channel.

7. The apparatus of claim 6, wherein the first fuel stream comprises a first purifier configured to purify the first input fuel.

8. The apparatus of claim 7, wherein the first input fuel is a reference fuel.

9. The apparatus of claim 1, comprising a second fuel stream configured to deliver a second input fuel of the plurality of input fuels to the fuel flow channel.

10. The apparatus of claim 9, comprising a component configured to change the concentration of impurities in the second input fuel.

11. The apparatus of claim 10, wherein the component is a second purifier.

12. The apparatus of claim 11, comprising a plurality of intermediate fuel streams extending from a plurality of intermediate positions on the second purifier configured to deliver a plurality of partially purified input fuels to the fuel flow channel.

13. The apparatus of claim 11, wherein the second purifier comprises a series of purifiers each configured to remove a different contaminant from the second input fuel.

14. The apparatus of claim 13, comprising a plurality of intermediate fuel streams extending from between each of the series of purifiers configured to deliver a plurality of partially purified input fuels to the fuel flow channel.

15. The apparatus of claim 9, wherein the second fuel stream is configured to deliver the second input fuel to one or more fuel cells.

16. The apparatus of claim 15, wherein the control system is configured to prevent delivery of the second input fuel to the one or more fuel cells when the second input fuel has a quality below a specified value.

17. The apparatus of claim 1, comprising an oxidizer flow channel adjacent to the cathode.

18. The apparatus of claim 17, wherein the oxidizer flow channel comprises a continuous path.

19. The apparatus of claim 1, wherein the fuel flow channel is divided into a plurality of sections.

20. The apparatus of claim 19, wherein each of the plurality of input fuels is received by a different section of the fuel flow channel.

21. The apparatus of claim 1, comprising a vent configured to remove inert contaminants from the electrochemical sensor.

22. A method for comparing fuel sources comprising:
supplying a plurality of input fuels to a plurality of locations on a fuel flow channel of an electrochemical sensor;
measuring the current produced by the plurality of input fuels in a plurality of segments of an anode or a cathode of the electrochemical sensor; and
determining the current density in each of the plurality of segments.

23. The method of claim 22, comprising detecting differences in the current densities of the plurality of segments to compare the quality of the plurality of input fuels.

24. The method of claim 22, comprising producing an output dependent on the quality of the input fuels.

25. The method of claim 23, wherein detecting differences in the current densities of the plurality of segments to compare the quality of the plurality of input fuels comprises using rule base programming methods or machine learning techniques.
